SEQUENCE LISTING (18 sequences)

SEQ ID NO: 1 - DNA, 1932 bp, Escherichia coli
  FtsH metalloprotease
atggcgaaaaacctaatactctggctggtcattgccgttgtgctgatgtcagtattccagagctttgggcccagcgagtctaatggccgtaaggtggattactctaccttcctacaagaggtcaataacgaccaggttcgtgaagcgcgtatcaacggacgtgaaatcaacgttaccaagaaagatagtaaccgttataccacttacattccggttcaggatccgaaattactggataacctgttgaccaagaacgtcaaggttgtcggtgaaccgcctgaagaaccaagcctgctggcttctatcttcatctcctggttcccgatgctgttgctgattggtgtctggatcttcttcatgcgtcaaatgcagggcggcggtggcaaaggtgccatgtcgtttggtaagagcaaagcgcgcatgctgacggaagatcagatcaaaacgacctttgctgacgttgcgggctgcgacgaagcaaaagaagaagttgctgaactggttgagtatctgcgcgagccgagccgcttccagaaactcggcggtaagatcccgaaaggcgtcttgatggtcggtcctccgggtaccggtaaaacgctgctggcgaaagcgattgcaggcgaagcgaaagttccgttctttactatctccggttctgacttcgtagaaatgttcgtcggtgtgggtgcatcccgtgttcgtgacatgttcgaacaggcgaagaaagcggcaccgtgcatcatctttatcgatgaaatcgacgccgtaggccgccagcgtggcgctggtctgggcggtggtcacgatgaacgtgaacagactctgaaccagatgctggttgagatggatggcttcgaaggtaacgaaggtatcatcgttatcgccgcgactaaccgtccggacgttctcgacccggccctgctgcgtcctggccgtttcgaccgtcaggttgtggtcggcttgccagatgttcgcggtcgtgagcagatcctgaaagttcacatgcgtcgcgtaccattggcacccgatatcgacgcggcaatcattgcccgtggtactcctggtttctccggtgctgacctggcgaacctggtgaacgaagcggcactgttcgctgctcgtggcaacaaacgcgttgtgtcgatggttgagttcgagaaagcgaaagacaaaatcatgatgggtgcggaacgtcgctccatggtgatgacggaagcgcagaaagaatcgacggcttaccacgaagcgggtcatgcgattatcggtcgcctggtgccggaacacgatccggtgcacaaagtgacgattatcccacgcggtcgtgcgctgggtgtgactttcttcttgcctgagggcgacgcaatcagcgccagccgtcagaaactggaaagccagatttctacgctgtacggtggtcgtctggcagaagagatcatctacgggccggaacatgtatctaccggtgcgtccaacgatattaaagttgcgaccaacctggcacgtaacatggtgactcagtggggcttctctgagaaattgggtccactgctgtacgcggaagaagaaggtgaagtgttcctcggccgtagcgtagcgaaagcgaaacatatgtccgatgaaactgcacgtatcatcgaccaggaagtgaaagcactgattgagcgtaactataatcgtgcgcgtcagcttctgaccgacaatatggatattctgcatgcgatgaaagatgctctcatgaaatatgagactatcgacgcaccgcagattgatgacctgatggcacgtcgcgatgtacgtccgccagcgggctgggaagaaccaggcgcttctaacaattctggcgacaatggtagtccaaaggctcctcgtccggttgatgaaccgcgtacgccgaacccgggtaacaccatgtcagagcagttaggcgacaag

SEQ ID NO: 2 - protein, 644 aa, Escherichia coli
  FtsH metalloprotease
MAKNLILWLVIAVVLMSVFQSFGPSESNGRKVDYSTFLQEVNNDQVREARINGREINVTKKDSNRYTTYIPVQDPKLLDNLLTKNVKVVGEPPEEPSLLASIFISWFPMLLLIGVWIFFMRQMQGGGGKGAMSFGKSKARMLTEDQIKTTFADVAGCDEAKEEVAELVEYLREPSRFQKLGGKIPKGVLMVGPPGTGKTLLAKAIAGEAKVPFFTISGSDFVEMFVGVGASRVRDMFEQAKKAAPCIIFIDEIDAVGRQRGAGLGGGHDEREQTLNQMLVEMDGFEGNEGIIVIAATNRPDVLDPALLRPGRFDRQVVVGLPDVRGREQILKVHMRRVPLAPDIDAAIIARGTPGFSGADLANLVNEAALFAARGNKRVVSMVEFEKAKDKIMMGAERRSMVMTEAQKESTAYHEAGHAIIGRLVPEHDPVHKVTIIPRGRALGVTFFLPEGDAISASRQKLESQISTLYGGRLAEEIIYGPEHVSTGASNDIKVATNLARNMVTQWGFSEKLGPLLYAEEEGEVFLGRSVAKAKHMSDETARIIDQEVKALIERNYNRARQLLTDNMDILHAMKDALMKYETIDAPQIDDLMARRDVRPPAGWEEPGASNNSGDNGSPKAPRPVDEPRTPNPGNTMSEQLGDK

SEQ ID NO: 3 - DNA, 1911 bp, Bacillus subtilis
  FtsH metalloprotease
atgaatcgggtcttccgtaataccattttttatttacttattttattagtagtaatcggggttgtgagctacttccagacctcaaatccgaaaacagaaaatatgtcgtacagtacgttcatcaaaaacctggatgacgggaaagttgatagcgtatcggttcagcctgtcagaggtgtttatgaggtaaaagggcagctgaaaaactacgacaaagatcaatactttttgactcatgttcctgaaggaaagggagcagaccagatatttaacgctttgaaaaagacagacgtaaaggttgagcccgcgcaagaaacaagcggatgggtgacgttcctgacgaccatcatcccatttgtcattatctttattctgtttttcttcctgctcaatcaggctcaaggcggcggcagccgtgtcatgaactttggcaagagtaaagcgaagctgtatacagaggaaaagaaacgcgtcaaatttaaagacgttgcaggggctgacgaagaaaagcaagaacttgttgaagttgttgagtttctgaaagatccccgcaagtttgccgagctcggcgccagaataccgaaaggcgtgcttttagtcggacctccgggtaccggtaaaacattgcttgccaaggcttgtgcaggagaagccggcgtacctttcttcagcatcagcggatctgatttcgttgaaatgtttgtaggggtcggtgcttcccgtgtgcgtgacttgtttgaaaatgcgaaaaagaatgcgccttgtttgatcttcattgatgaaattgacgcagtcggacgccagcgtggcgctggtctcggcggtggacacgatgaacgtgaacagacgctaaaccaattgcttgttgaaatggacggattcagcgctaatgaaggaattatcatcattgctgcgacgaaccgtgcggacatcttggacccagccttacttcgtccgggacgttttgaccgtcaaatcacagtggaccgcccagatgtcattggccgtgaagctgtattgaaagtccatgcgagaaacaaaccgctggatgaaacggttaacctaaaatcaattgccatgagaacaccaggcttctcaggcgctgacttagaaaacctcttgaatgaagctgcgcttgtagcggctcgtcaaaacaagaaaaaaatcgatgcgcgtgatattgacgaagcgacggaccgtgtaattgccggacccgctaagaagagccgcgttatctccaagaaagaacgcaatatcgtggcttatcacgaaggcggacacaccgttatcggtctcgttttagatgaggcagatatggttcataaagtaacgattgttcctcggggccaggctggcggttatgctgttatgctgccaagagaagaccgttatttccaaacaaagccggagctgcttgataaaattgtcggcctcttgggcggacgtgttgctgaagagattatcttcggtgaagtcagcacaggggcgcacaatgacttccagcgtgcgacgaatattgcaagacgaatggttacagaattcggtatgtcagaaaaactgggaccgttgcaatttggacagtctcagggcggtcaggtattcttaggccgtgatttcaacaacgaacagaactacagtgatcaaatcgcttacgaaattgatcaggaaattcagcgcatcatcaaagaatgttatgagcgtgcgaaacaaatcctgactgaaaatcgtgacaagcttgaattgattgcccaaacgcttctgaaagttgaaacgcttgacgctgaacaaatcaaacaccttatcgatcatggaacattacctgagcgtaatttctcagatgatgaaaagaacgatgatgtgaaagtaaacattctgacaaaaacagaagaaaagaaagacgatacgaaagag

SEQ ID NO: 4 - protein, 637 aa, Bacillus subtilis
  FtsH metalloprotease
MNRVFRNTIFYLLILLVVIGVVSYFQTSNPKTENMSYSTFIKNLDDGKVDSVSVQPVRGVYEVKGQLKNYDKDQYFLTHVPEGKGADQIFNALKKTDVKVEPAQETSGWVTFLTTIIPFVIIFILFFFLLNQAQGGGSRVMNFGKSKAKLYTEEKKRVKFKDVAGADEEKQELVEVVEFLKDPRKFAELGARIPKGVLLVGPPGTGKTLLAKACAGEAGVPFFSISGSDFVEMFVGVGASRVRDLFENAKKNAPCLIFIDEIDAVGRQRGAGLGGGHDEREQTLNQLLVEMDGFSANEGIIIIAATNRADILDPALLRPGRFDRQITVDRPDVIGREAVLKVHARNKPLDETVNLKSIAMRTPGFSGADLENLLNEAALVAARQNKKKIDARDIDEATDRVIAGPAKKSRVISKKERNIVAYHEGGHTVIGLVLDEADMVHKVTIVPRGQAGGYAVMLPREDRYFQTKPELLDKIVGLLGGRVAEEIIFGEVSTGAHNDFQRATNIARRMVTEFGMSEKLGPLQFGQSQGGQVFLGRDFNNEQNYSDQIAYEIDQEIQRIIKECYERAKQILTENRDKLELIAQTLLKVETLDAEQIKHLIDHGTLPERNFSDDEKNDDVKVNILTKTEEKKDDTKE

SEQ ID NO: 5 - DNA, 25 bp, Artificial Sequence
  Description of Artificial Sequence E. coli FtsH forward primer
atggcgaaaaacctaatactctggc

SEQ ID NO: 6 - DNA, 23 bp, Artificial Sequence
  Description of Artificial Sequence E. coli FtsH reverse primer
tcacttgtcgcctaactgctctg

SEQ ID NO: 7 - DNA, 30 bp, Artificial Sequence
  Description of Artificial Sequence Staphylococcus FtsH forward primer
atgcagaaagcttttcgcaatgtgctagtt

SEQ ID NO: 8 - DNA, 30 bp, Artificial Sequence
  Description of Artificial Sequence Staphylococcus FtsH reverse primer
ttatttattgtctgggtgatttggatcgta

SEQ ID NO: 9 - DNA, 14 bp, Artificial Sequence
  Description of Artificial Sequence Promotor consensus sequence
ttgcnnnnnnttgc

SEQ ID NO: 10 - DNA, 14 bp, Artificial Sequence
  Description of Artificial Sequence Promotor consensus sequence with ctr-1 mutation
ttgcnnnnnnttgt

SEQ ID NO: 11 - DNA, 49 bp, Artificial Sequence
  Description of Artificial SequencePromotor fragment
tcgttgcgtttgtttgcacgaaccatatgtaagtatttccttagataac

SEQ ID NO: 12 - DNA, 49 bp, Artificial Sequence
  Description of Artificial SequencePromotor fragment
ttcttgcgtgtaattgcggagactttgcgatgtacttgacacttcagga

SEQ ID NO: 13 - DNA, 33 bp, Artificial Sequence
  Description of Artificial Sequence PCR primer
gacgagctcaagctttgatctgcgacttatcaa

SEQ ID NO: 14 - DNA, 33 bp, Artificial Sequence
  Description of Artificial Sequence PCR primer
cgcggatccccttcccgagtaacaaaaaaacaa

SEQ ID NO: 15 - DNA, 27 bp, Artificial Sequence
  Description of Artificial Sequence PCR primer
gagggatccatgaccatgattacggat

SEQ ID NO: 16 - DNA, 27 bp, Artificial Sequence
  Description of Artificial Sequence PCR primer
ctcgatatcctgcaccatcgtctgctc

SEQ ID NO: 17 - DNA, 30 bp, Artificial Sequence
  Description of Artificial Sequence PCR primer
cacgatatcctgctgatgaagcagaacaac

SEQ ID NO: 18 - DNA, 33 bp, Artificial Sequence
  Description of Artificial Sequence PCR primer
gacggtaccaagcttttatttttgacaccagac

(19) United States
(12) Patent Application Publication
(10) Pub. No.: US 2003/0027251 A1
(43) Pub. Date: Feb. 6, 2003

(54) Assays for inhibitors of FtsH

(76) Inventors: Goutam Das, Bangalore (IN); Camille Deluca-Flaherty, Palo Alto, CA (US); Marc Navre, Belmont, CA (US); Nita Roy, Bangalore (IN); Suresh Subramani, San Diego, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 09/948,137

(22) Filed: Sep. 5, 2001

Related U.S. Application Data
(63) Continuation of application No. PCT/US00/04659, filed on Feb. 22, 2000.
(60) Provisional application No. 60/122,911, filed on Mar. 5, 1999.

(51) Int. Cl. C12P 21/02; C12N 1/21

(52) U.S. Cl. 435/69.1; 435/252.33; 435/252.3

(57) ABSTRACT

This invention provides a bacterial system and assay for detecting and quantifying activity of bacterial growth regulator, FtsH. The bacteria include three expression cassettes. An FtsH expression cassette comprises a first promoter operatively linked to a nucleotide sequence encoding FtsH. A transcriptional regulator expression cassette comprises a second promoter operatively linked to a nucleotide sequence encoding a transcriptional regulator which regulates the activity of a third promoter, wherein the transcriptional regulator is a substrate of FtsH. A reporter expression cassette comprises the third promoter operatively linked to a reporter gene. The activity of FtsH can be read out as a positive expression of the reporter gene. The invention also provides an assay for compounds that modulate the expression of FtsH. The assay involves contacting the recombinant bacterial cell with the agent, and determining whether the agent modulates the expression of the reporter gene.

### BACKGROUND OF THE INVENTION

This invention relates to the fields of antibiotics, drug screening and molecular biology. More particularly, this invention is directed to model systems and screening methods for compounds that inhibit the growth of E. coli and, more specifically, compounds that inhibit the activity of FtsH.

The development of antibiotics against pathogenic organisms is a medically and commercially important activity. E. coli is a bacterium that can be pathogenic. It is known as a contaminant of meat, especially ground beef. The development of antibiotics against E. coli would have a positive impact on public health.

One strategy in the development of antibiotics is to identify genes that are essential to the growth of the pathogen, and screen agents that inhibit the activity of these genes or their products. One such gene in E. coli is FtsH.

FtsH is a zinc-containing metalloprotease belonging to the AAA (ATPase associated with various activities) family of ATPases which are ubiquitous in bacteria, fungi and higher organisms. (Y. T. Akiyama et al. (1994) “Involvement of FtsH in protein assembly into and through the membrane. I. Mutations that reduce retention efficiency of a cytoplasmic reporter” J. Biol. Chem. 269:5218-5224; Y. T. Akiyama et al. (1994) “Involvement of FtsH in protein assembly into and through the membrane. II. Dominant mutations affecting FtsH functions” J. Biol. Chem. 269:5225-5229; Z. Ge et al. (1996) “Sequencing, expression, and genetic characterization of the Helicobacter pylori ftsH encoding a protein homologous to members of a novel putative ATPase family” J. Bacteriol. 178:6151-6157; E. Lysenko et al. (1997) “Characterization of the ftsH gene of Bacillus subtilis” Microbiology 143 971-978.) FtsH is an essential gene in E. coli and in H. pylori (Akiyama et al., supra, Ge et al., supra). The gene also is known to exist in other bacteria and in yeast. The FtsH protein has two membrane-spanning domains and is located in the inner membrane of E. coli. (Y. Akiyama et al. (1996) “FtsH (HflB) is an ATP-dependent protease selectively acting on SecY and some other membrane proteins” J. Biol. Chem. 271:31196-31201.)

FtsH is involved in variety of cellular processes such as degradation of the heat-shock transcription factor σ³². (T. Tomayasu et al. (1995) “Escherichia coli FtsH is a membrane-bound, ATP-dependent protease which degrades the heat-shock transcription factor σ³²” EMBO J. 14:2551-2560; C. Herman et al. (1995), “Degradation of stigma 32, the heat shock regulator in Escherichia coli, is governed by HflB,” Proc. Natl. Acad. Sci. USA, 92:3516-20.) It also is involved in the stability of mRNA in bacteria. (R. F. Wang et al. (1998) “Escherichia coli mrsC is an allele of hflB, encoding a membrane-associated ATPase and protease that is required for mRNA decay,” J. Bacteriol., 180:1929-38.)

In addition to these essential cellular processes, FtsH also functions as a switch between lysis and lysogeny for phage λ. (Y. Shotland et al. (1997) “Proteolysis of the phage λ cII regulatory protein by FtsH (HflB) of Escherichia coli” Mol. Microbiol. 24:1303-1310; Y. Akiyama (1998), “Roles of the periplasmic domain of Escherichia coli FtsH (hflB) in protein interactions and activity modulation.” J. Biol. Chem., 273:22326-33.) This is because λcII, which is involved in the transition from lysis to lysogeny, is one of the substrates for the FtsH protease. (Shotland et al., supra.) The cII protein activates the λ promoters P_{RE}, P_{I} and P_{AQ}, which are involved in the expression of the λ repressor and of other inhibitor proteins essential for the conversion of λ phage-infected cells to lysogeny. (M Obuchowski et al. (1997) “Stability of CII is a key element in the cold stress response of bacteriophage λ infection” J. Bacteriol. 179:5987-5991; Shotland et al., supra.) FtsH also degrades the λcIII protein which stabilizes λcII and E. coli σ³² proteins, thus inhibiting their degradation by FtsH. (Herman et al. (1997) “The HflB protease of Escherichia coli degrades its inhibitor λcIII” J. Bacteriology 179:358-363.)

### SUMMARY OF THE INVENTION

This invention provides a bacterial system and method to screen for agents that modulate the activity of the bacterial protein FtsH. The system involves a two-part circuit. In a first part of the circuit, a transcriptional regulator, preferably an activator, positively regulates the expression of a reporter gene. In a second part of the circuit, FtsH negatively regulates the activity of the transcriptional regulator. Thus, increasing levels of FtsH expression result in decreased levels of reporter gene expression, and decreased levels of FtsH expression result in increased levels of reporter gene expression.

The system can be used to test agents for their ability to modulate the activity of FtsH. A bacterium that harbors the completed circuit is exposed to the test agent. If the test agent inhibits the activity of FtsH, the circuit responds with increased expression of the reporter gene.

This system provides advantages for screening compounds. First, it is a positive read-out system: Inhibitors of FtsH are identified by detecting expression of the reporter gene. Second, it is sensitive: Inhibitors of FtsH are potential antibiotics. However, rather than detecting bacterial death, which is a crude measurement, this system can detect fine differences in FtsH inhibition as a function of reporter expression. Third, it is fast: The response of the reporter gene to decreased activity of FtsH occurs in a very short time. Fourth, it allows high through-put: It is a cell based assay which does not require any purification steps, and the response of a reporter gene can be easily measured in a small volume of cells, resulting in miniaturization of the process, as well as the simultaneous analysis of many concentrations of a given FtsH inhibitor, or of many such inhibitors.

In one aspect, this invention provides a recombinant bacterial cell. The cell comprises three expression cassettes. A first FtsH expression cassette comprises an expression control sequence operatively linked to a nucleotide sequence encoding FtsH. A second expression cassette comprises a second expression control sequence operatively linked to a nucleotide sequence encoding transcriptional regulator which regulates the expression of a third promoter, and the transcriptional regulator is proteolytically inactivated by FtsH. A third expression cassette comprises the third promoter operatively linked to a sensitive and easily assayed nucleotide sequence encoding a reporter gene.

In another aspect, this invention provides a method for determining whether an agent modulates the activity of FtsH. The method involves contacting a recombinant bacterial cell of this invention with the test compound, and determining whether the compounds causes a change in the expression of the reporter gene. When the transcriptional regulator is a transcriptional activator, the compounds that inhibit activity of FtsH will result in increased expression of the reporter gene. Since the compounds are applied to the outside of living bacteria, the test evaluates both the entry of the compound into bacteria and the inhibition of intracellular FtsH by the compound.

### BRIEF DESCRIPTION OF THE DRAWINGS

FIG. 1 depicts the circuit of this invention. A transcriptional regulator positively regulates the expression of a reporter gene, whose expression can be measured. The activity of the transcriptional regulator is negatively regulated by the product of a controller gene, whose activity is to tested. In this example, the transcriptional regulator is λC_{II}, expressed in pBR322 under the control of the inducible p_{tac} promoter. λC_{II} activates the PRE promoter, which is operatively linked to the reporter gene, β-gal, also on the same pBR322-based plasmid. The activity of this circuit is regulated by the controller gene product, which functions as a sort of biological rheostat. In this case, the controller gene is FtsH. FtsH is expressed from a pACYC184-based plasmid under the control of the inducible P_{BAD} promoter. FtsH is a protease. λC_{II} is a substrate of FtsH. Decreases in FtsH activity increase the activity of λC_{II} which, in turn, increase the expression of the reporter gene, β-gal. The activity of agents to modulate the activity of FtsH can be measured by the positive read-out of their impact on β-gal expression, measured in an activity assay.

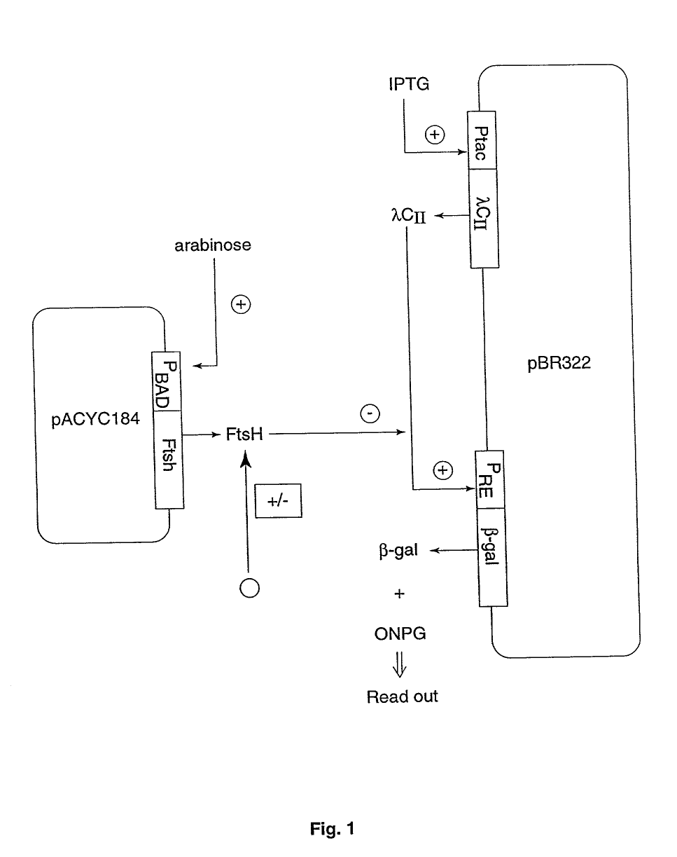

### DETAILED DESCRIPTION OF THE INVENTION

### I. DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); THE GLOSSARY OF GENETICS, 5TH ED., R. Rieger et al. (eds.), Springer Verlag (1991); and Hale & Marham, THE HARPER COLLINS DICTIONARY OF BIOLOGY (1991). As used herein, the following terms have the meanings ascribed to them unless specified otherwise.

“Nucleic acid” refers to a polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. Thus, the term includes nucleotide polymers in which the nucleotides and the linkages between them include non-naturally occurring synthetic analogs, such as, for example and without limitation, phosphorothioates, phosphoramidates, methyl phosphonates, chiral-methyl phosphonates, 2-O-methyl ribonucleotides, peptide-nucleic acids (PNAs), and the like. Such polynucleotides can be synthesized, for example, using an automated DNA synthesizer. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe nucleotide sequences: the left-hand end of a single-stranded polynucleotide sequence is the 5′end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an MRNA is referred to as the “coding strand”; sequences on the DNA strand having the same sequence as an mRNA transcribed from that DNA and which are located 5′ to the 5′-end of the RNA transcript are referred to as “upstream sequences”; sequences on the DNA strand having the same sequence as the RNA and which are 3′ to the 3′ end of the coding RNA transcript are referred to as “downstream sequences.”

“cDNA” refers to a DNA that is complementary or identical to an MRNA, in either single stranded or double stranded form.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Recombinant nucleic acid” refers to a nucleic acid having nucleotide sequences that are not naturally joined together. An amplified or assembled recombinant nucleic acid may be included in a suitable vector, and the vector can be used to transform a suitable host cell. A host cell that comprises the recombinant nucleic acid is referred to as a “recombinant host cell.” The gene is then expressed in the recombinant host cell to produce, e.g., a “recombinant polypeptide.” A recombinant nucleic acid may serve a non-coding function (e.g., promoter, origin of replication, ribosome-binding site, etc.) as well.

“Expression control sequence” refers to a nucleotide sequence in a polynucleotide that regulates the expression (transcription and/or translation) of a nucleotide sequence operatively linked thereto. “Operatively linked” refers to a functional relationship between two parts in which the activity of one part (e.g., the ability to regulate transcription) results in an action on the other part (e.g., transcription of the sequence). Expression control sequences can include, for example and without limitation, sequences of promoters (e.g., inducible or constitutive), enhancers, transcription terminators, a start codon (i.e., ATG), splicing signals for introns, and stop codons. “Promoter” refers to an expression control sequence that directs transcription of a nucleic acid. Promoters include necessary sequences near the start site of transcription, such as, for example, a TATA element. Promoters also can include distal enhancer or repressor elements.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in vitro expression system. Expression vectors include all those known in the art, such as cosmids, plasmids (e.g., naked or contained in liposomes) and viruses that incorporate the recombinant polynucleotide.

“Expression cassette” refers to a recombinant nucleic acid construct comprising an expression control sequence operatively linked to an expressible nucleotide sequence.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer. The term “protein” typically refers to large polypeptides. The term “peptide” typically refers to short polypeptides. Conventional notation is used herein to portray polypeptide sequences: the left-hand end of a polypeptide sequence is the amino-terminus; the right-hand end of a polypeptide sequence is the carboxyl-terminus.

“Allelic variant” refers to any of two or more polymorphic forms of a gene occupying the same genetic locus. Allelic variations arise naturally through mutation, and may result in phenotypic polymorphism within populations. Gene mutations can be silent (no change in the encoded polypeptide) or may encode polypeptides having altered amino acid sequences. “Allelic variants” also refer to cDNAs derived from mRNA transcripts of genetic allelic variants, as well as the proteins encoded by them.

“Small organic molecule” refers to organic molecules of a size comparable to those organic molecules generally used in pharmaceuticals. The term excludes organic biopolymers (e.g., proteins, nucleic acids, etc.). Preferred small organic molecules range in size up to about 5000 Da, up to about 2000 Da, or up to about 1000 Da.

“Chemical library” refers to a collection of compounds of different structures. Generally, the compounds will fall into the same class of chemical compounds, e.g., DNA, polypeptides, benzodiazepines, etc. Such libraries frequently are referred to as “combinatorial libraries.”

“Transcriptional regulator” refers to a protein that regulates the activity of a promoter. “Transcriptional activator” refers to a transcriptional regulator that up-regulates the activity of a promoter.

A transcriptional regulator is a substrate of FtsH if FtsH diminishes the ability of the regulator to regulate the activity of a promoter.

“Reporter gene” refers to a nucleic acid comprising a nucleotide sequence that encodes a detectable transcription product. The detectable transcription product can be an RNA or a protein resulting from translation of the RNA.

“FtsH” refers to a zinc-containing metalloprotease belonging to the AAA family of ATPases and genes that encode it, including allelic variants. This includes FtsH of E. coli and homologs of it in other bacteria, archeabacteria and yeast. FtsH genes can be identified by a high degree of sequence identity. The nucleotide and amino acid sequence of E. coli FtsH are:

| | |
|---|---|
| atggcgaaaa acctaatact ctggctggtc attgccgttg tgctgatgtc | (SEQ ID NO:1) |
| agtattccag agctttgggc ccagcgagtc taatggccgt aaggtggatt | |
| actctacctt cctacaagag gtcaataacg accaggttcg tgaagcgcgt | |
| atcaacggac gtgaaatcaa cgttaccaag aaagatagta accgttatac | |
| cacttacatt ccggttcagg atccgaaatt actggataac ctgttgacca | |
| agaacgtcaa ggttgtcggt gaaccgcctg aagaaccaag cctgctggct | |
| tctatcttca tctcctggtt cccgatgctg ttgctgattg gtgtctggat | |
| cttcttcatg cgtcaaatgc agggcggcgg tggcaaaggt gccatgtcgt | |
| ttggtaagag caaagcgcgc atgctgacgg aagatcagat caaaacgacc | |
| tttgctgacg ttgcgggctg cgacgaagca aaagaagaag ttgctgaact | |
| ggttgagtat ctgcgcgagc cgagccgctt ccagaaactc ggcggtaaga | |
| tcccgaaagg cgtcttgatg gtcggtcctc cgggtaccgg taaaacgctg | |
| ctggcgaaag cgattgcagg cgaagcgaaa gttccgttct ttactatctc | |
| cggttctgac ttcgtagaaa tgttcgtcgg tgtgggtgca tcccgtgttc | |
| gtgacatgtt cgaacaggcg aagaaagcgg caccgtgcat catctttatc | |
| gatgaaatcg acgccgtagg ccgccagcgt ggcgctggtc tgggcggtgg | |
| tcacgatgaa cgtgaacaga ctctgaacca gatgctggtt gagatggatg | |
| gcttcgaagg taacgaaggt atcatcgtta tcgccgcgac taaccgtccg | |
| gacgttctcg acccggccct gctgcgtcct ggccgtttcg accgtcaggt | |
| tgtggtcggc ttgccagatg ttcgcggtcg tgagcagatc ctgaaagttc | |
| acatgcgtcg cgtaccattg gcacccgata tcgacgcggc aatcattgcc | |
| cgtggtactc ctggtttctc cggtgctgac ctggcgaacc tggtgaacga | |
| agcggcactg ttcgctgctc gtggcaacaa acgcgttgtg tcgatggttg | |
| agttcgagaa agcgaaagac aaaatcatga tgggtgcgga acgtcgctcc | |
| atggtgatga cggaagcgca gaaagaatcg acggcttacc acgaagcggg | |
| tcatgcgatt atcggtcgcc tggtgccgga acacgatccg gtgcacaaag | |
| tgacgattat cccacgcggt cgtgcgctgg gtgtgacttt cttcttgcct | |
| gagggcgacg caatcagcgc cagccgtcag aaactggaaa gccagatttc | |
| tacgctgtac ggtggtcgtc tggcagaaga gatcatctac gggccggaac | |
| atgtatctac cggtgcgtcc aacgatatta aagttgcgac caacctggca | |
| cgtaacatgg tgactcagtg gggcttctct gagaaattgg gtccactgct | |
| gtacgcggaa gaagaaggtg aagtgttcct cggccgtagc gtagcgaaag | |
| cgaaacatat gtccgatgaa actgcacgta tcatcgacca ggaagtgaaa | |
| gcactgattg agcgtaacta taatcgtgcg cgtcagcttc tgaccgacaa | |
| tatggatatt ctgcatgcga tgaaagatgc tctcatgaaa tatgagacta | |
| tcgacgcacc gcagattgat gacctgatgg cacgtcgcga tgtacgtccg | |
| ccagcgggct gggaagaacc aggcgcttct aacaattctg gcgacaatgg | |
| tagtccaaag gctcctcgtc cggttgatga accgcgtacg ccgaacccgg | |
| gtaacaccat gtcagagcag ttaggcgaca ag | |
| MAKNLILWLV IAVVLMSVFQ SFGPSESNGR KVDYSTFLQE VNNDQVREAR | (SEQ ID NO:2) |
| INGREINVTK KDSNRYTTYI PVQDPKLLDN LLTKNVKVVG EPPEEPSLLA | |
| SIFISWFPML LLIGVWIFFM RQMQGGGGKG AMSFGKSKAR MLTEDQIKTT | |
| FADVAGCDEA KEEVAELVEY LREPSRFQKL GGKIPKGVLM VGPPGTGKTL | |
| LAKATAGEAK VPFFTISGSD FVEMFVGVGA SRVRDMFEQA KKAAPCIIFI | |
| DEIDAVGRQR GAGLGGGHDE REQTLNQMLV EMDGFEGNEG IIVIAATNRP | |
| DVLDPALLRP GRFDRQVVVG LPDVRGREQI LKVHMRRVPL APDIDAAIIA | |
| RGTPGFSGAD LANLVNEAAL FAARGNKRVV SMVEFEKAKD KIMMGAERRS | |
| MVNTEAQKES TAYHEAGHAT IGRLVPEHDP VHKVTIIPRG RALGVTFFLP | |
| EGDAISASRQ KLESQISTLY GGRLAEEITY GPEHVSTGAS NDIKVATNLA | |
| RNMVTQWGFS EKLGPLLYAE EEGEVFLGRS VAKAKHMSDE TARIIDQEVK | |
| ALIERNYNRA RQLLTDNMDI LHAMKDALMK YETIDAPQID DLMARRDVRP | |
| PAGWEEPGAS NNSGDNGSPK APRPVDEPRT PNPGNTMSEQ LGDK | |

The nucleotide and amino acid sequence of Bacillus subtilis FtsH are:

| | |
|---|---|
| atgaatcggg tcttccgtaa taccattttt tatttactta ttttattagt | (SEQ ID NO:3) |
| agtaatcggg gttgtgagct acttccagac ctcaaatccg aaaacagaaa | |
| atatgtcgta cagtacgttc atcaaaaacc tggatgacgg gaaagttgat | |
| agcgtatcgg ttcagcctgt cagaggtgtt tatgaggtaa aagggcagct | |
| gaaaaactac gacaaagatc aatacttttt gactcatgtt cctgaaggaa | |
| agggagcaga ccagatattt aacgctttga aaaagacaga cgtaaaggtt | |
| gagcccgcgc aagaaacaag cggatgggtg acgttcctga cgaccatcat | |
| cccatttgtc attatcttta ttctgttttt cttcctgctc aatcaggctc | |
| aaggcggcgg cagccgtgtc atgaactttg gcaagagtaa agcgaagctg | |
| tatacagagg aaaagaaacg cgtcaaattt aaagacgttg caggggctga | |
| cgaagaaaag caagaacttg ttgaagttgt tgagtttctg aaagatcccc | |
| gcaagtttgc cgagctcggc gccagaatac cgaaaggcgt gcttttagtc | |
| ggacctccgg gtaccggtaa aacattgctt gccaaggctt gtgcaggaga | |
| agccggcgta cctttcttca gcatcagcgg atctgatttc gttgaaatgt | |
| ttgtaggggt cggtgcttcc cgtgtgcgtg acttgtttga aaatgcgaaa | |
| aagaatgcgc cttgtttgat cttcattgat gaaattgacg cagtcggacg | |
| ccagcgtggc gctggtctcg gcggtggaca cgatgaacgt gaacagacgc | |
| taaaccaatt gcttgttgaa atggacggat tcagcgctaa tgaaggaatt | |
| atcatcattg ctgcgacgaa ccgtgcggac atcttggacc cagccttact | |
| tcgtccggga cgttttgacc gtcaaatcac agtggaccgc ccagatgtca | |
| ttggccgtga agctgtattg aaagtccatg cgagaaacaa accgctggat | |
| gaaacggtta acctaaaatc aattgccatg agaacaccag gcttctcagg | |
| cgctgactta gaaaacctct tgaatgaagc tgcgcttgta gcggctcgtc | |
| aaaacaagaa aaaaatcgat gcgcgtgata ttgacgaagc gacggaccgt | |
| gtaattgccg gacccgctaa gaagagccgc gttatctcca agaaagaacg | |
| caatatcgtg gcttatcacg aaggcggaca caccgttatc ggtctcgttt | |
| tagatgaggc agatatggtt cataaagtaa cgattgttcc tcggggccag | |
| gctggcggtt atgctgttat gctgccaaga gaagaccgtt atttccaaac | |
| aaagccggag ctgcttgata aaattgtcgg cctcttgggc ggacgtgttg | |
| ctgaagagat tatcttcggt gaagtcagca caggggcgca caatgacttc | |
| cagcgtgcga cgaatattgc aagacgaatg gttacagaat tcggtatgtc | |
| agaaaaactg ggaccgttgc aatttggaca gtctcagggc ggtcaggtat | |
| tcttaggccg tgatttcaac aacgaacaga actacagtga tcaaatcgct | |
| tacgaaattg atcaggaaat tcagcgcatc atcaaagaat gttatgagcg | |
| tgcgaaacaa atcctgactg aaaatcgtga caagcttgaa ttgattgccc | |
| aaacgcttct gaaagttgaa acgcttgacg ctgaacaaat caaacacctt | |
| atcgatcatg gaacattacc tgagcgtaat ttctcagatg atgaaaagaa | |
| cgatgatgtg aaagtaaaca ttctgacaaa aacagaagaa aagaaagacg | |
| atacgaaagag | |
| MNRVFRNTIF YLLILLVVIG VVSYFQTSNP KTENMSYSTF IKNLDDGKVD | (SEQ ID NO:4) |
| SVSVQPVRGV YEVKGQLKNY DKDQYFLTHV PEGKGADQIF NALKKTDVKV | |
| EPAQETSGWV TFLTTIIPFV IIFILFFFLL NQAQGGGSRV NNFGKSKAKL | |
| YTEEKKRVKF KDVAGADEEK QELVEVVEFL KDPRKFAELG ARIPKGVLLV | |
| GPPGTGKTLL AKACAGEAGV PFFSISGSDF VEMFVGVGAS RVRDLFENAK | |
| KNAPOLIFID EIDAVGRQRG AGLGGGHDER EQTLNQLLVE MDGFSANEGI | |
| IIIAATNRAD ILDPALLRPG RFDRQITVDR PDVIGREAVL KVHARNKPLD | |
| ETVNLKSIAM RTPGFSGADL ENLLNEAALV AARQNKKKID ARDIDEATDR | |
| VIAGPAKKSR VISKKERNIV AYHEGGHTVI GLVLDEADMV HKVTIVPRGQ | |
| AGGYAVMLPR EDRYFQTKPE LLDKTVGLLG GRVAEEIIFG EVSTGAKNDF | |
| QRATNIARRM VTEFGMSEKL GPLQFGQSQG GQVFLGRDFN NEQNYSDQIA | |
| YEIDQEIQRI IKECYERAKQ ILTENPDKLE LIAQTLLKVE TLDAEQIKHL | |
| IDHGTLPERN FSDDEKNDDV KVNILTKTEE KKDDTKE | |

The nucleotide and amino acid sequence of Staphylococcus FtsH is presented in EP 0 801 132 (Sarginson et al.).

### II. RECOMBINANT HOST CELL—BACTERIAL SYSTEM

This invention provides a recombinant bacterial host cell useful for screening modulators (usually inhibitors) of FtsH. The recombinant bacterium of this invention comprises three expression cassettes; (1) an FtsH expression cassette for expressing FtsH, (2) a transcriptional regulator expression cassette for expressing a transcriptional regulator that also is a substrate of FtsH, and (3) a reporter expression cassette comprising a promoter that is regulated by the transcriptional regulator, and which is operatively linked to a reporter gene.

A. FtsH Expression Cassette

A first expression cassette comprises an expression control sequence operatively linked with a nucleotide sequence encoding FtsH.

The FtsH can be any bacterial or yeast FtsH for which one seeks to identify modulators. However, it is preferable to use an FtsH that is native to the bacterial system in use. For example, in an E. coli bacterial system, wild type E. coli FtsH or allelic variants are preferable. E. coli FtsH can be obtained by amplification of E. coli DNA using the following primers:

Forward primer: 5′-atggcgaaaa acctaatact ctggc-3′ (SEQ ID NO:5)

Reverse primer: 5′-tcacttgtcg cctaactgct ctg- 3′ (SEQ ID NO:6) These primers would yield a sequence from start to stop codon.

The gene encodes a protein of 644 amino acids having a predicted mass of 70.7 kDa. Nucleic acids encoding E. coli FtsH can be identified by several characteristics including size (about 2 kb), characteristic restriction map, sequence or by the fact that it expresses a protein that is cross-reactive with a rabbit antibody specific to FtsH.

The practitioner also can use FtsH genes from other bacteria and yeast. FtsH has been identified in Bacillus subtilis (N. Ogasawara et al. (1994) DNA Res. 1:1-14), Lactococcus lactis (D. Nilsson et al. (1994) Microbiology 140:2601-2610), Staphylococcus aureus (G. Sarginson et al., EP 0 801 132 (Oct. 15, 1997), Saccharomyces cerevisiae P. E. Thorsness et al. ('993) Molec. and Cell. Biol. 13:5418-5426) and S. typhimurium.

A primer pair that can be used to amplify sequences encoding Staphylococcus FtsH is:

5′ Primer: 5′-atgcagaaag cttttcgcaa tgtgctagtt-3′ (SEQ ID NO:7)

3′ Primer: 5′-ttatttattg tctgggtgat ttggatcgta-3′ (SEQ ID NO:8)

FtsH genes from all bacterial species share sufficient homology that one can design degenerate primers of about 20-25 nucleotides in length, based on the conservation of the known DNA sequences of this gene from various bacterial species. The DNA fragment obtained by the use of these PCR primers on a genomic DNA template from that bacterium could then be used to isolate the full-length FtsH gene from a genomic library of DNA fragments from that bacterial species.

The nucleic acid segment encoding FtsH is operatively linked to an expression control sequence that can affect transcription of the gene. In the practice of the assays of this invention, the levels of expression of FtsH and of the transcriptional regulator, which FtsH cleaves, must be calibrated against each other so that a decrease in FtsH expression can be manifested in an increase in regulator expression measurable by the activity of the regulated promoter operatively linked to the reporter gene. Thus, it is preferable to use an inducible promoter to regulate expression of FtsH. In particular, the promoter preferably is regulated by the addition of an inducing compound, rather than by, for example, changes in temperature. Such promoters are more responsive and more easily controlled. Useful regulable promoters include P_{tac}, lac and P_{BAD}. P_{tac} and lac can be regulated by the addition of IPTG. The P_{BAD} can be regulated by the addition of arabinose. These promoters are well known and can be easily obtained from various vendors or by PCR using the sequence of the E. coli genome which is published at http://mol.genes.nig.ac.jp/ecoli/ecwcgi.exe?CMD=GEN_RETRIEVE and http//www.pasteur.fr/Bio/Colibri.html.

B. Transcriptional Regulator Expression Cassette

A second expression cassette comprises an expression control sequence operatively linked with a nucleotide sequence encoding a transcriptional regulator that is also a substrate of FtsH. The transcriptional regulator is preferably a transcriptional activator. The transcriptional regulator functions in the circuit to regulate the expression of a reporter gene.

λC_{II} is a preferred transcriptional activator that is proteolytically inactivated by FtsH. λC_{II} regulates the activity of the P_{RE}, P_{I} and P_{AQ} promoters. C_{II} is a well-characterized protein from bacteriophage λ. The nucleotide sequence of the λC_{II} is the segment between bp 38360 and 38650 (orf 97) in the bacteriophage genome (Genbank accession # J02459 or M17233) and can be obtained by PCR with suitably-designed primers.

One also can use modified versions of C_{II}, that recognize different sequences in target promoters. For example, promoters recognized by C_{II} contain the consensus sequence 5′-T-T-G-C-N₆-T-T-G-C-3′ (SEQ ID NO:9). The ctr-1 mutation of C^{II}, alters this recognition sequence to 5′-T-T-G-C- N₆-T-T-G-T-3′ (SEQ ID NO: 10). However, it has not effect on promoter activity (e.g., P_{RE}).

σ³² (also called htpR) another transcriptional activator that is proteolytically inactivated by FtsH. The σ³² factor is a subunit of E. coli RNA polymerase. The sequence of E. coli π³² is described in Landrick et al. (1984) “Nucleotide sequence of the heat shock regulatory gene of E. coli suggests its protein product may be a transcription factor,” Cell 38:175-182. The sequence of π³² is located between nucleotides 3595544 and 3594693 at map location 77.5 in the E. coli genome, and can be obtained by PCR with suitably-designed primers.

As discussed above, the expression level of the transcriptional regulator should be tuned in coordination with the expression level of the FtsH gene. Over-expression of the transcriptional regulator results in continuous expression of the reporter gene. In this case, changes in FtsH activity have little or no effect on reporter gene expression. Under-expression of the transcriptional activator results in too little expression of the reporter gene, so that even large decreases in FtsH activity will not result in detectable increases in reporter gene expression.

Proper tuning of transcriptional activator expression can be achieved in two ways. First, the host cell should include many copies of the gene. This can be accomplished by including the expression cassette on a high copy number plasmid.

Second, the nucleotide sequence of the transcriptional regulator should be operatively linked to a regulable expression control sequence. The same kinds of regulable promoters useful for controlling expression of the FtsH-linked promoter also are useful for regulating expression of the transcriptional regulator. However, the transcriptional regulator expression cassette should include a different regulable promoter than the FtsH expression cassette. In this way, the two expression cassettes can be tuned individually.

C. Reporter Expression Cassette

A third expression cassette comprises an expression control sequence regulated by the transcriptional regulator which is operatively linked with a nucleotide sequence encoding a reporter gene.

The expression control sequence to which the reporter gene is operatively linked comprises a promoter whose activity is regulated by the transcriptional regulator. In gram negative bacteria, such as E. coli and S. typhimurium, cII regulates the P_{RE}, P_{I} and P_{AQ} promoters. Nucleic acids encoding the P_{RE}, P_{I} and P_{AQ} promoters can be obtained as follows. A DNA fragment containing λP_{RE} has the sequence 5′ TCGTTGCGTT TGTTTGCACG AACCATATGT AAGTATTTCC TTAGATAAC 3′ (SEQ ID NO:11). A DNA fragment containing λP_{I} has the sequence 5′ TTCTTGCGTG TAATTGCGGA GACTTTGCGA TGTACTTGAC ACTTCAGGA 3′ (SEQ ID NO: 12). P_{AQ} also contains the consensus sequence discussed above.

σ³² regulates the expression of heat shock gene promoters which can be used as the promoter of the reporter gene in this expression cassette. There are about twenty heat shock genes that are regulated by σ³² including, for example, lon, groEL and dnaK. Heat shock genes can be identified in Colibri web site discussed above. See also C. A. Gross, “Function and Regulation of Heat Shock Proteins,” Chapter 88 of ESCHERICHIA COLI AND SALMONELLA Second edition, F. C. Neidhardt, ed. (1996) ASM Press, Washington, D.C.

Preferred reporter genes have five characteristics. First, they are non-toxic to the cell. That is, their expression does not result in noticeable inhibition of cell growth or in cell death, nor should it offer a selective growth advantage to cells. Second, the reporter gene ordinarily should not be expressed by the cell, so that there is low background expression that might interfere with the sensitivity of the assay. Third, the reporter gene should be easily detectable, especially by the production of a visible signal. Fourth, changes in expression in the reporter gene should be detectably quickly. Fifth, the activity of the reporter should be quantifiable.

One preferred class of reporter genes are the fluorescent proteins, such as Aequorea green fluorescent proteins or mutants of it having different excitation or emission characteristics that fluoresce at different wavelengths. These proteins can be detected with fluorescent optics. Such proteins are described, for example, in U.S. Pat. No. 5,625,048 (Tsien et al.) and U.S. Pat. No. 5,804,387 (Cormack et al.).

Luciferase also is produces a visible light signal. (de Wet et al. (1987), “Firefly luciferase gene: Structure and expression in mammalian cells,” Mol. Cell. Biol. 7:725-737.)

β-galactosidase is a well known reporter gene. Its activity is easily detectable in an enzymatic assay. Simply, cells are lysed and exposed to a substrate, ONPG. In a few minutes the color reaction proceeds to detectability. Other substrates useful in this invention are those that, upon cleavage, yield a fluorescent product. One example is β-methylumbelliferyl beta-D-galactopyranoside (MUG).

In order to provide the best reading of the signal, it is preferable that the reporter gene expression cassette be located on a high copy number plasmid. Thus, the transcriptional regulator expression cassette and the reporter gene expression cassette can be on the same vector. This can be preferable, as two high copy number plasmids may be incompatible in a single cell.

D. Hosts And Vectors

The recombinant bacterial system of this invention includes three expression cassettes that create the test circuit. The expression cassettes are recombinant nucleic acids in which the nucleotide sequence to be expressed (FtsH, transcriptional regulator, reporter gene) is operatively linked with a non-native promoter. The recombinant nucleic acids can exist in the cell separate from the bacterial chromosome or integrated into it. Plasmids are the preferred free-standing recombinant vectors because they are easily introduced and rescued from bacterial cells. Other useful vectors include, for example, phage (e.g., λ) or transposons.

The expression cassettes can be on one or more than one vector. One variable in choosing a plasmid vector is copy number. It is preferable that the FtsH expression cassette be introduced on a low copy number plasmid. A low copy number plasmid is a plasmid that exists in about 5-10 copies per cell. Examples of low copy number vectors include pACYC184 or pACYC177 containing the origin of DNA replication from plasmid p15A. The transcriptional activator expression cassette and the reporter gene expression cassette preferably are introduced into the cell on high copy number plasmids. A high copy number plasmid is a plasmid that exists in at least 30 copies, usually 30 to 50 copies, per cell. Examples of high copy number vectors include pBR322, pUC19 and others containing the ColE1 origin of DNA replication. Thus, the transcriptional regulator expression cassette and the reporter gene expression cassette can be introduced on the same plasmid vector. However, it is preferable that neither of these expression cassettes is introduced on the same vector as the FtsH expression cassette.

The host cell is chosen so that the promoters and expressed nucleic acids that are parts of the circuit of this invention function in that cell. This is particularly true for the transcriptional regulator and the promoter whose expression it regulates. The function of these units depends on factors such as the particular RNA polymerase in the cell and the cytoplasmic environment of the cell. λC_{II} and σ32 and the promoters they regulate function best in E. coli. However, they also function well in other gram negative bacteria, such as S. typhimurium. They also are expected to function in gram positive bacteria, such as Staphylococcus.

One factor in choosing the host is the ability to test an agent for the ability to modulate the activity of that host's native FtsH. Agents that inhibit a particular FtsH are candidate antibiotics for that host.

Another factor in choosing the host is its permeability to introduced agents. The more easily accessible the host is to the agent, the more control one has in testing agents. Gram positive bacteria are more permeable to agents than gram negative bacteria. This is an advantage of using gram positive bacteria.

### III. ASSAYS FOR MODULATORS OF FtsH ACTIVITY

This invention provides methods of screening compounds to identify those that modulate FtsH activity. Such methods are useful for identifying candidate antibiotics against E. coli and other bacteria or single celled organisms that harbor FtsH.

Assays for modulators of biological activity generally involve administering the test agent to an assay system, and determining whether the agent alters the amount of the biological activity in the assay system. This determination generally involves measuring the amount of biological activity of the assay system resulting after administration of the test agent, and comparing that amount to a control or standard amount of biological activity. The control amount preferably reflects the biological activity of the assay system when no agent has been added. For example, the determination can involve performing a side-by-side comparison of biological activity with and without administration of the test compound. In another method, the practitioner can create a “standard curve” in which the system is exposed to varying amounts of the agent and the amount of biological activity is measured. The activity measurements are extrapolated to a zero amount of agent administration. In this way the amount of activity upon administration of the compound can be compared to the amount of activity when no agent is administered. The practitioner also can compare the amount of biological activity resulting from the administration of different amounts of the test agent. In this case one amount provides a “test” level of activity and the other amount provides a “control” level of activity. A difference between the test amount and the control amount indicates that the agent modulates biological activity. The comparison between test amounts of activity and control amounts can provide a simple “yes” or “no” answer to the question of whether the agent modulates activity. Alternatively, if the answer is “yes” that amount can be quantified. Modulation contemplates both up-regulation and down-regulation of activity.

This invention contemplates the testing of any chemical or biological agent in the activity assay. Thus, the “agent” can be a chemical compound (e.g., a small organic molecule or a bioorganic molecule), a mixture of chemical compounds, or an extract made from biological materials such as bacteria, plants, fungi, or animal cells or tissues. The system of this invention is useful for testing libraries of compounds by exposing different cultures of the recombinant bacteria to different agents in the library.

Assays for testing agents for the ability of a compound to modulate the activity of FtsH begin with cultivating a recombinant bacterial cell of this invention. Thus, the cell normally is cultivated under conditions usual conditions for growth, including proper temperature, nutrients, ionic environment, antibiotic etc. At a determined time, the FtsH gene and the transcriptional regulator gene are induced by activating the promoters to which they are operatively linked.

In large scale screenings, the bacteria can be deposited in microtiter plates or other small volume devices or in the form of a lawn of bacteria. In certain embodiments, a single cell can be cultured and tested. The agent is administered to the cell or culture. Usually, the agent will be delivered in varying amounts, in order to determine a level of modulation. The cell is then cultured for sufficient time for the reporter system to “develop.” Thus, depending upon the particular reporter system chosen, this time involves time for expression of the reporter gene and the manifestation of signal. When the reporter is a fluorescent protein, this time can be on the order of minutes. When the reporter is an enzyme, e.g., β-galactosidase, the assay will involve supplying substrate to the cells and allowing time for the enzyme to act on the substrate.

Then, the amount of expression of the reporter gene is measured by appropriate means. For example, fluorescent proteins can be detected by fluorimeter. Other color reactions can be measured by spectrometric analysis. The determination of whether a test agent modulates activity generally will involve comparing the amount of reporter gene expression as measured with a control amount.

Agents which prove to be modulators of FtsH in this assay can be further evaluated as prospective antibiotics.

### EXAMPLE

The following example is provided by way of illustration, not by way of limitation.

We created a recombinant E. coli of this invention in which the regulation of expression of a reporter gene attached to a λP_{RE} promoter by C_{II} was controlled by FtsH.

### I. MATERIALS AND METHODS

A. Bacterial Strains and Plasmids

E. coli strain MC4100 (lacZ⁻) was used in all the lacZ expression studies. Routine cloning was done in E. coli DHA. The standard cloning vectors used were pBR322 (New England Biolabs), pBSKS+ (Stratagene) and pACYC 184 (New England Biolabs). Regulated expression of FtsH was achieved by cloning the FtsH gene in the vector pAR-FtsH under the control of the arabinose-inducible P_{BAD} promoter. The plasmids used in this study are described in Table I.

TABLE I
Bacterial strains and plasmids used in this study

| Strain or | Relevant genotype | Source or |
|---|---|---|
| Plasmid | or characteristics | reference |
| Strains | | |
| DH5α | recA1 end A1 hsd R17 SupE GyrA96 relA1 | Lab |
| | Δ(lac ZYA-arg F) U169 (φ8odlacZ Δ M15) | collection |
| MC4100 | F-AraD139(argF-lac)U169 rpsL150(Str r) | Lab |
| | relA1flbB5301 deoC1 pts F25 rbsR | collection |
| SYKD001 | MC4100/pSYN013 | This study |
| SYKD002 | MC4100/pSYN017 | This study |
| SYKD003 | MC4100/pSYN018 | This study |
| SYKD004 | MC4100/pSYN019 | This study |
| SYKD005 | SYKD004/pSYN020 | This study |
| SYKD006 | SYKD004/pSYN021 | This study |
| SYKD007 | SYKD004/pAR-FtsH | This study |
| SYKD008 | SYKD004/pAR-FtsH(E415A) | This study |
| Plasmids: | | |
| pBSKS+ | AmpR; Col EI origin | Stratagene |
| pBR322 | AmpR; Col EI origin | NEB |
| pACYC184 | TetR; CmR; origin from p15A | NEB |
| pHG333 | Carries the ptac-cII fragment (including the | Gift |
| | lacIq) | |
| pSYN013 | 2.2 kb LacIq + ptac-cII fragment into SaII site | This study |
| | of pBR322 | |
| pSYN014 | λ PRE region into SacI(5') and BamHI(3') | This study |
| | sites of pBSKS | |
| pSYN015 | N-terminal 1.0 kb fragment of LacZ into | This study |
| | BamHI(5') and EcoRV (3') sites of pSYN014 | |
| pSYN016 | C-terminal 2.0 kb fragment of LacZ into | This study |
| | EcoRV(5') and KpnI (3') sites of pSYN015 | |
| pSYN017 | 3.3 kb λ PRE-LacZ fragment into HindIII site | This study |
| | of pBR322 (clockwise orientation) | |
| pSYN018 | 3.3 kb λ PRE-LacZ fragment into HindIII site | This study |
| | of pBR322 (anti-clockwise) | |
| pSYN019 | 2.2 kb LacIq + ptac-cII fragment into SaII | This study |
| | site of pSYN017 | |
| pSYN020 | 2.0 kb ftsh into EcoRI site of pACYC184 | This study |
| pSYN021 | 2.0 kb ftsh(E415A) into EcoRI site of | This study |
| | pACYC184 | |

B. Construction of Plasmids

A 2.2 kb SalI fragment (including the lacl^{q} gene) containing the λ cII gene located downstream from the P_{tac}, promoter was excised from pHG333 and cloned into the SalI site of pBR322 to generate pSYN013.

The λP_{RE}-lacZ construct was made as follows: the λP_{RE} promoter region spanning the λ coordinates 38480 to 38210, was amplified using polymerase chain reaction (PCR) with primers λP_{RE}1 (forward ^{5′}GAC GAG CTC AAG CTT TGA TCT GCG ACT TAT CAA^{3′} (SEQ ID NO:13) ) and λP_{RE}2 (reverse ^{5′}CGC GGA TCC CCT TCC CGA GTA ACA AAA AAA CAA^{3′} (SEQ ID NO:14)) using λ DNA as the template. The conditions for PCR amplification were: 94° C. (30 seconds), 60° C. (30 seconds) and 72° C. (30 seconds). The PCR product was cloned between the SacI (5′) and the BamHI (3′) sites of pBSKS+ to generate pSYN014.

To clone the lacZ downstream from P_{RE}, the lacZ gene was cloned in two steps by PCR amplification using E. coli K12 chromosomal DNA as the template. In the first step, the N-terminal 1.0 kb fragment of lacZ was amplified using PCR with the primers Lac Z1a (forward ^{5′}GAG GGA TCC ATG ACC ATG ATT ACG GAT^{3′} (SEQ ID NO:15)) and Lac Z1b (reverse ^{5′}CTC GAT ATC CTG CAC CAT CGT CTG CTC³′(SEQ ID NO: 16)) under the conditions 94° C. (30 seconds), 61° C. (30 seconds) and 72° C. (1 minute). The PCR product lacz1 was cloned between the BamHI and EcoRV sites of pSYN014 to obtain the plasmid pSYN015. The 3′-region of lacZ (lacz2) was amplified by PCR using the primers LacZ 2a (forward ^{5′}CAC GAT ATC CTG CTG ATG AAG CAG AAC AAC^{3′}(SEQ ID NO:17)) and LacZ 2b (reverse ^{5′}GAC GGT ACC AAG CTT TTA TTT TTG ACA CCA GAC³ (SEQ ID NO:18)) using the following conditions: 94° C. (30 seconds), 59° C. (45 seconds) and 72° C. (1 minute). The 2.0 kb lacZ 2 product was cloned between the EcoRV and KpnI sites of pSYN015 to generate pSYN016. The resulting plasmid contains the entire 3.0 kb lacZ gene under the control of the λP_{RE}. The PCR primers were designed so that the entire P_{RE}-lacZ segment could be excised as a HindIII fragment from pSYN016. This 3.3 kb HindIII fragment containing P_{RE} -lacZ was then subcloned into the HindIII site of pBR322 to obtain pSYN017 (clockwise orientation) and pSYN018 (anticlockwise orientation). The 2.2 kb SalI fragment from pSYN013 containing Lacl^{q} and P_{TAQ}-CII was then subcloned into the SalI site of pSYN017 to yield pSYN019. All the PCR-generated gene fragments were sequenced to confirm the nucleotide sequences.

The 2.0 kb FtsH gene and the protease-deficient mutant, ftsh (E415A), were isolated from pSYN002 and pSYN007 and cloned into the EcoRI site of pACYC184 to generate the plasmids pSYN020 and pSYN021, respectively. Expression of FtsH under the control of tightly-regulated P_{BAD} promoter was obtained by constructing the plasmid pAR-FtsH, where the FtsH gene was cloned in the P_{BAD} vector downstream of the arabinose-inducible promoter P_{BAD} between the NCI (N-terminal) and HindIII (C-terminal) sites (Roy et al.). Similarly, pAR-FtsH(E415A) was constructed by cloning FtsH(E415A) (Roy et al) in the PBAD vector between the same sites as above.

C. Assay for β-galactosidase Activity

Overnight cultures were inoculated in LB (with appropriate antibiotic selection: ampicillin 50 μg/ml; chloramphenicol 20 μg/ml; tetracycline 5 μ/ml) at an initial OD₆₀₀ of 0.04. After ˜2 hours at 37° C. and continuous shaking at 200 rpm (OD₆₀₀˜0.3) the cultures were induced with 500 μM IPTG and 0.2% arabinose, when necessary. When the cultures were grown in the minimal medium (+0.2% glucose), cells were induced at an OD₆₀₀˜0.4. Following induction, the OD₆₀₀ was recorded at different time points and the lacZ expression was quantified by determining the β-galactosidase activity. The assay method followed was essentially as described by Miller (1972). Briefly, 100 μl of induced cells was added to 900 μl of Z-buffer (Na₂HPO₄ 60 mM, NaH₂PO₄ 40 mM, KCl 10 mM, MgSO₄ 1 mM, β-mercaptoethanol 50 mM) and lysed by the addition of 2 drops of chloroform and 1 drop of 0.1% SDS followed by vortexing. After the completion of lysis, 200 μl of ONPG (o-nitrophenyl-β-D-galactopyranoside; 6 mg/ml in 0.1M phosphate buffer, pH 7.0) was added and the color reaction was allowed to proceed for 2-5 minutes. The reaction was stopped by adding 500 μl of 1M Na₂CO₃. The cell debris was spun down and the supernatant was subjected to spectrophotometric analysis at 420 nm.

β-galactosidase activity was calculated using the formula:

$$Units = 1000 \times \frac{O {D}_{420}}{t \times V \times O {D}_{600}}$$

t=time of development of assay (mins) before stopping the reaction

V=volume of cells (ml) used for assay

### II. RESULTS

A. Effect of Orientation of Cloning of P_{RE}-lacZ on the Expression of lacZ

The lacZ gene under the control of λP_{RE} promoter was cloned in both orientations in pBR322. The strains SYKD002 and SYKD003 harboring pSYN017(clockwise) and pSYN018 (anti-clockwise) respectively, were tested for background levels of lacZ expression. There was a significant level of lacZ expression from SYKD003, while the levels of expression from SYKD002 remained similar to that of the host strain, MC4100 (Table II). Such occurrences of fortuitous activation of λP_{RE}-driven genes as seen in the strain SYKD003, have been known to occur depending upon the orientation of cloning. The strain SYKD002 was chosen for further studies due to its lower background levels of LacZ.

TABLE II
Effect of λP_{RE} promoter orientation on the expression of lacZ.

| Strain | β-gal units^{a} |
|---|---|
| MC4100 | 100 |
| SYKD002 (clockwise) | 103 |
| SYKD003 (anti-clockwise) | 1368 |

^{a}Cultures were assayed for β-galactosidase as described by Miller (1972)

B. Effect of cII on the Expression of lacZ from P_{RE}-lacZ

To study the cII-dependent expression of lacZ, plasmid pSYN019 (containing the lacl^{q}/P_{tac}-cII fragment cloned into pSYN017) was used to transform MC4100 cells resulting in the strain SYKD004. These transformants were grown in LB broth at 37° C. and checked for β-galactosidase expression after cII induction with IPTG. As shown in Table III, there was more than a 20-fold increase in the expression level of β-galactosidase in the induced cultures compared to the uninduced ones, demonstrating the cII-dependent expression of lacZ.

TABLE III
The effect of cII activated λP_{RE} on LacZ expression

| | Uninduced | IPTG-Induced^{a} | Fold^{b} |
|---|---|---|---|
| Strain | (β-gal units) | (β-gal units) | Induction |
| SYKD002(lacZ) | 245 | 272 | — |
| SYKD004(lacZ/cII) | 311 | 6415 | 21 |
| ^{a}IPTG-induced cultures were assayed for β-galactosidase activity as described by J.H. Miller (1972) | | | |
| # EXPERIMENTS IN MOLECULAR GENETICS. Cold Spring Harbor Laboratory, Cold Spring Harbor, NY. | | | |

^{b}Fold activity was obtained by dividing the β-gal units of the induced cultures by that of the uninduced.

### C. Effect of FtsH on the cII-dependent Expression Level of β-galactosidase

1) Expression of ftsH Under the Control of its' Native Promoter

To determine the effect of FtsH on cII-dependent lacZ expression, the ftsH gene along with its native promoter, was cloned into pACYC184 yield pSYN020. The latter was used to transform SYKD004 resulting in the strain SYKD005. The strain SYKD005 therefore harbors two compatible plasmids which under appropriate induction conditions can express FtsH, cII and LacZ simultaneously. The negative control pSYN021 (pACYC harboring FtsH-E415A, a protease-deficient mutant of ftsH with its native promoter) was similarly transformed into SYK004 to yield the strain SYK006. The resultant strains SYK005 and SYKD006 were grown in LB at 37° C. in the presence of ampicillin and tetracycline and cII was induced with IPTG for 2 hrs as described in Materials and Methods. As shown in Table IV, the fold induction of β-galactosidase was reduced to half when cII was induced with IPTG in the presence of FtsH. This reduction in the expression of lacZ was not observed in the presence of FtsH (E415A). The reduction in the β-galactosidase activity for SYKD005 is presumed to be due to the in vivo proteolytic degradation of the λP_{RE} activator protein cII by FtsH.

TABLE IV
The effect of FtsH on cII-activated λP_{RE}-LacZ expression.

| | Uninduced | Induced^{a} | |
|---|---|---|---|
| Strain | (β-gal units) | (β-gal units) | Fold^{b} |
| SYKD004(lacZ/cII) | 317 | 6710 | 21 |
| SYKD005(lacZ/cII/ftsH)^{c} | 450 | 4400 | 10 |
| SYKD006(lacZ/cII/ftsH-E415A)^{d} | 399 | 10310 | 26 |
| ^{a}IPTG-induced cultures were assayed for β-galactosidase activity as described by Miller (1972). | | | |
| ^{b}Fold activity was obtained by dividing the β-galactosidase units of the induced cultures by that of | | | |
| # the uninduced. | | | |

^{c}FtsH and FtsH(E415A) are expressed under the control of the native FtsH promoter.

2) Expression of ftsH Under the Control of the Regulable Promoter P_{BAD}

To determine the effect of varying levels of FtsH in cII-dependent LacZ expression, ftsH was cloned downstream of the stringently-regulated P_{BAD} promoter (pAR-FtsH). This expression plasmid was used to transform SYKD004 resulting in the strain SYKD007. Similarly, as a control, pAR-FtsH(E415A) was transformed into SYKD004 to yield SYKD008. The cells were grown in minimal medium at 37° C. and were induced with varying amounts of arabinose for the expression of FtsH. As shown in Table V, inducing the strain SYKD008 at different concentrations of arabinose did not have any effect on the levels of β-galactosidase being produced, indicating that the proteolytically-inactive form of FtsH has no effect on cII. However, in SYKD007 the level of β-galactosidase appeared to be directly correlated to the amount of arabinose used for induction reflecting the modulation by FtsH of the LacZ activator, cII.

TABLE V
Modulating effect of arabinose-induced FtsH expression on
β-galactosidase activity

% β-galactosidase activity^{a}

| Arabinose | SYKD007^{b} | SYKD008^{c} |
|---|---|---|
| concentration (%) | (lacZ/cII/ftsH) | (lacZ/cII/ftsH-E415A) |
| 0 | 100 | 100 |
| 0.002 | 100 | 67.5 |
| 0.02 | 100 | 23 |
| 0.2 | 100 | 20 |
| ^{a}%β-galactosidase activity calculated by normalizing the β-galactosidase units of the uninduced | | |
| # cultures to 100%. | | |

^{b,c}FtsH and FtsH(E415A) expressed under the control of pBAD promoter.

The present invention provides novel materials and methods for detecting modulators of FtsH activity. While specific examples have been provided, the above description is illustrative and not restrictive. Many variations of the invention will become apparent to those skilled in the art upon review of this specification. The scope of the invention should, therefore, be determined not with reference to the above description, but instead should be determined with reference to the appended claims along with their full scope of equivalents.

All publications and patent documents cited in this application are incorporated by reference in their entirety for all purposes to the same extent as if each individual publication or patent document were so individually denoted. By their citation of various references in this document Applicants do not admit that any particular reference is “prior art” to their invention.

What is claimed is:

1. A recombinant bacterial cell comprising:
a) an FtsH expression cassette comprising a first promoter operatively linked to a nucleotide sequence encoding FtsH;
b) a transcriptional regulator expression cassette comprising a second promoter operatively linked to a nucleotide sequence encoding a transcriptional regulator which regulates the activity of a third promoter, wherein the transcriptional regulator is a substrate of FtsH; and
c) a reporter expression cassette comprising the third promoter operatively linked to a reporter gene.

2. The recombinant bacterial cell of claim 1 wherein the FtsH is an E. coli FtsH.

3. The recombinant bacterial cell of claim 2 wherein the substrate is λC_{II}.

4. The recombinant bacterial cell of claim 2 wherein the substrate is σ32.

5. The recombinant bacterial cell of claim 3 wherein the bacterial cell is E. coli.

6. The recombinant bacterial cell of claim 3 wherein the bacterial cell is Salmonella.

7. The recombinant bacterial cell of claim 5 wherein the first and second promoters are inducible promoters.

8. The recombinant bacterial cell of claim 5 wherein the third promoter is P_{RE}.

9. The recombinant bacterial cell of claim 5 wherein the third promoter is selected from the group consisting of P_{I} and P_{AQ}.

10. The recombinant bacterial cell of claim 5 wherein at the first and second expression cassettes are comprised on high copy number plasmids.

11. The recombinant bacterial cell of claim 3 wherein the reporter gene is selected from the group consisting of β-galactosidase, luciferase and a fluorescent protein.

12. The recombinant bacterial cell of claim 7 wherein the one of the first and second promoters is P_{BAD}.

13. The recombinant bacterial cell of claim 7 wherein the one of the first and second promoters is P_{tac}.

14. A method for determining whether an agent modulates the activity of FtsH comprising:
a) contacting a bacterial cell with the agent, wherein the cell:
i) expresses FtsH;
ii) expresses a transcriptional regulator that regulates the activity of a target promoter, wherein the transcriptional regulator is a substrate of FtsH; and
iii) comprises an expression cassette which comprises the target promoter operatively linked to a reporter gene; and
b) determining whether contact with the agent modulates the expression of the reporter gene;
whereby modulation of the expression of the reporter gene provides a determination that the compound modulates the activity of FtsH.

15. The method of claim 14 wherein the FtsH is an E. coli FtsH.

16. The method of claim 14 comprising contacting a plurality of cells each with a different agent, and recording agents that modulate the activity of FtsH.

17. The method of claim 15 wherein the transcriptional regulator is λC_{II}.

18. The method of claim 15 wherein the transcriptional regulator is σ³².

19. The method of claim 17 wherein the target promoter is selected from the group consisting of P_{RE}, P_{I} and P_{AQ}.

20. The method of claim 19 wherein the FtsH and the transcriptional regulator are expressed under the control of first and second inducible promoters, respectively, and wherein the method further comprises inducing the expression of FtsH and the transcriptional regulator.

21. The method of claim 19 wherein the reporter gene is selected from the group consisting of β-galactosidase, luciferase and a fluorescent protein.

22. The method of claim 20 wherein one of the first and second inducible promoters is P_{BAD}.

23. The method of claim 20 wherein one of the first and second inducible promoters is P_{tac}.

24. The method of claim 16 wherein different agents are a combinatorial library of small organic molecules.
